(19) United States
(12) Patent Application Publication
(10) Pub. No.: US 2025/0354898 A1
(43) Pub. Date: Nov. 20, 2025

(54) PICKING DEVICE

(71) Applicant: SHIMADZU CORPORATION, Kyoto (JP)

(72) Inventors: Takumi YAMADA, Kyoto (JP); Jun SHIMADA, Kyoto (JP); Momoyo YAMAKAWA, Kyoto (JP)

(73) Assignee: SHIMADZU CORPORATION, Kyoto (JP)

(21) Appl. No.: 19/194,961

(22) Filed: Apr. 30, 2025

(30) Foreign Application Priority Data
May 14, 2024 (JP) 2024-078366
Nov. 19, 2024 (JP) 2024-201816

(51) Int. Cl. G01N 1/14

(52) U.S. Cl. CPC G01N 1/14; G01N 2001/1418

(57) ABSTRACT

In a picking device configured to insert a pipet tip or similar sampling tool in a tilted posture into a container and to sample a sampling target from the same container, a picking device is provided which can avoid the situation in which an area from which the sampling target cannot be sampled occurs within the container. The picking device includes: a placement table having a placement surface on which a container is to be placed; a placement table rotation mechanism configured to rotate the placement table about a rotation axis which is a virtual axis orthogonal to the placement surface and passing through the center of the placement surface; a rod-shaped or tube-shaped sampling tool having a tip end configured to be inserted into the container, in a tilted posture to the rotation axis; and a sampling tool moving mechanism configured to move the sampling tool.

### TECHNICAL FIELD

The present invention relates to a picking device for sampling a cell or similar sampling target from a container.

### BACKGROUND ART

Cell-picking devices for assisting the operation of sampling a specific cell from a dish or similar container and transferring it to another container have conventionally been known (e.g., see Patent Literature 1). For example, a cell-picking device includes a microscope having an XY stage and a suction device configured to suction a cell from a dish or similar container placed on the XY stage and eject it into another container (e.g., a predetermined well on a microplate). The suction device is provided with a suction arm to which a pipet tip can be attached as well as a driver configured to move the suction arm and control the same arm to perform the suctioning and ejecting operations.

In the process of sampling a cell from a dish or similar container by the previously described type of cell-picking device, the three-dimensional position of the suction arm is adjusted by means of the driver, and the two-dimensional position of the dish or similar container in the X-Y plane (i.e., horizontal plane) is adjusted by means of the XY stage so that the tip end of the pipet tip attached to the suction arm comes close to the cell to be sampled. In this state, the suctioning operation using the suction arm is performed to suction the cell into the pipet tip.

### CITATION LIST

### Patent Literature

Patent Literature 1: JP 7255687 B

### SUMMARY OF INVENTION

### Technical Problem

In the previously described type of cell-picking device, when the tip end of the pipet tip is to be inserted into the dish, the pipet tip and the suction arm need to be tilted to the vertical axis so that they will not interfere with the illuminator, lens tube or other parts of the microscope located above the dish. However, inserting the pipet tip in a tilted posture into the dish in this manner means that an area within which the tip end of the pipet tip cannot be positioned (i.e., an area from which cells cannot be sampled) occurs near a portion of the circumferential wall of the dish within the inner area of the dish.

The present invention has been developed in view of the previously described problem. In a picking device configured to insert a pipet tip or similar sampling tool in a tilted posture into a container and to sample a sampling target from the same container, the objective of the present invention is to avoid the situation in which an area from which the sampling target cannot be sampled occurs within the container.

### Solution to Problem

A picking device according to the present invention developed for solving the previously described problem includes:
a placement table having a placement surface on which a container is to be placed;
a placement table rotation mechanism configured to rotate the placement table about a rotation axis which is a virtual axis orthogonal to the placement surface and passing through the center of the placement surface;
a rod-shaped or tube-shaped sampling tool having a tip end configured to be inserted into the container, in a tilted posture to the rotation axis; and
a sampling tool moving mechanism configured to move the sampling tool.

### Advantageous Effects of Invention

In the picking device according to the present invention having the previously described configuration, the placement table on which a container that contains the target to be sampled (“sampling target”) is placed can be rotated about the rotation axis orthogonal to the placement surface by means of the placement table rotation mechanism. Therefore, when the sampling tool is inserted in a tilted posture into the container by the sampling tool moving mechanism in order to sample the sampling target from the container, the situation can be avoided in which an area from which the sampling target cannot be sampled occurs within the container.

### BRIEF DESCRIPTION OF DRAWINGS

FIG. 1 is a perspective view of a cell-picking device according to one embodiment of the present invention.

FIG. 2 is a block diagram showing the configuration of the main components of a controller of the aforementioned cell-picking device.

FIG. 3 is a flowchart showing an operation of the aforementioned cell-picking device.

FIG. 4 is a plan view showing a dish and a pipet tip observed from above, for explaining an arrangeable area and a non-arrangeable area in the aforementioned embodiment.

FIG. 5 is a sectional view at the arrowed line A-A in FIG. 4.

FIG. 6 is a model diagram showing a dish and a pipet tip observed from one side, for explaining a method for determining an arrangeable area and a non-arrangeable area.

FIG. 7 is a model diagram showing the dish and the pipet tip in FIG. 6 as observed from above, for explaining a method for determining an arrangeable area and a non-arrangeable area.

FIG. 8 is a model diagram in which a plurality of virtual tip-end positions set in the X-Y plane are represented by a plurality of circles.

FIG. 9 is a model diagram in which the motions of the stage and the rotary table in a cell-sampling process are shown in time series.

FIG. 10 is a block diagram showing the configuration of the main components of a controller of a cell-picking device according to another embodiment of the present invention.

FIG. 11 is a flowchart showing the procedure for moving the table based on commands from a user received through a move command receiver in the aforementioned embodiment.

FIG. 12 is a flowchart showing the procedure for moving the table based on commands from a user received through a target position indication receiver in the aforementioned embodiment.

FIG. 13 is a perspective view of a cell-picking device according to still another embodiment of the present invention.

FIG. 14 is a block diagram showing the configuration of the main components of a controller of the cell-picking device according to the aforementioned embodiment.

### DESCRIPTION OF EMBODIMENTS

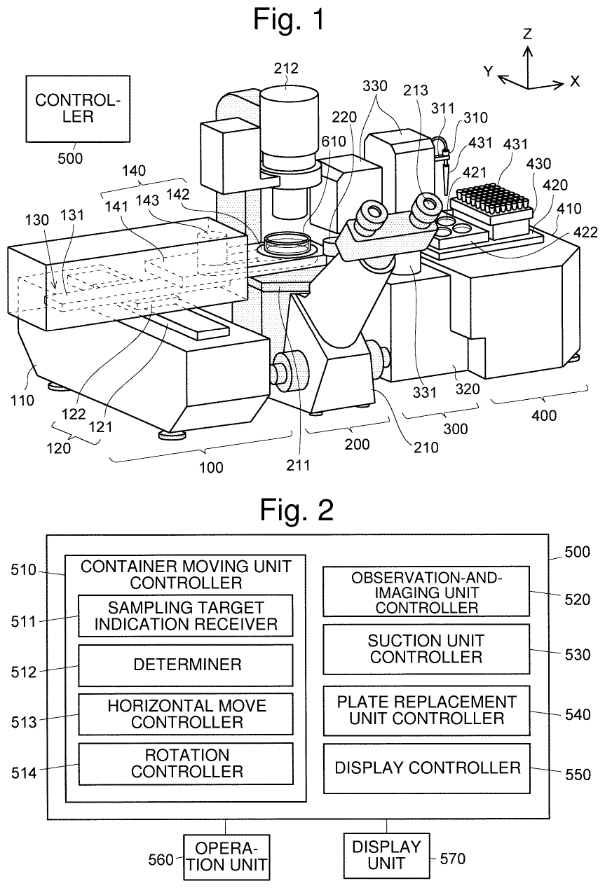

Modes for carrying out the present invention are hereinafter described with reference to the drawings. FIG. 1 is a perspective view of a cell-picking device according to one embodiment of the present invention. FIG. 2 is a block diagram showing the configuration of the main components of a controller 500 included in the same cell-picking device.

The cell-picking device according to the present embodiment includes a container moving unit 100, observation-and-imaging unit 200, suction unit 300, plate replacement unit 400, and controller 500. Hereinafter, the right-left, front-back and up-down directions are defined so that the arrowed directions of the X, Y and Z axes in FIG. 1 correspond to the rightward, backward and upward directions, respectively. As shown in FIG. 1, the container moving unit 100 and the suction unit 300 are arranged on the immediate left and right sides of the observation-and-imaging unit 200, respectively, and the plate replacement unit 400 is arranged on the immediate right side of the suction unit 300. It should be noted that the positional relationship of those units is not limited to this form; for example, the positional relationship of these units may be laterally reversed.

The container moving unit 100 includes a base 110 as well as a first moving mechanism 120 placed on the base 110, a second moving mechanism 130 placed on the first moving mechanism 120, and a table 140 placed on the second moving mechanism 130. The first and second moving mechanisms 120 and 130 correspond to the “placement table moving mechanism” in the present invention. The first moving mechanism 120 includes a first rail 121 fixed to the upper surface of the base 110 and extending in the front-back direction, a first slider 122 configured to be slidable along the first rail 121, and a first driving mechanism (not shown) including a linear motor and other components (not shown) for driving the first slider 122. The second moving mechanism 130 includes a second rail 131 fixed to the upper surface of the first slider 122 and extending in the right-left direction, a second slider (not shown) configured to be slidable along the second rail 131, and a second driving mechanism (not shown) including a linear motor and other components (not shown) for driving the second slider. The table 140 includes a base member 141 fixed to the upper surface of the second slider, a disk-shaped rotary table 142 (which corresponds to the “placement table” in the present invention) provided at the tip end of the base member 141, and a rotation mechanism 143 (which corresponds to the “placement table rotation mechanism”) provided in the base member 141 and configured to rotate the rotary table 142 about a vertical axis passing through the center of this table (this axis corresponds to the “rotation axis” in the present invention). The upper surface of the rotary table 142 serves as the placement surface on which a dish 610 (which corresponds to the “container” in the present invention) containing cells is to be placed. The placement surface has a recess into which the dish 610 is to be fitted. In the rotary table 142, at least an area of located immediately beneath the dish 610 (i.e., the central area) is made of a translucent material. The rotation mechanism 143 has a rotary motor as well as a rotation transmission mechanism consisting of a belt or gears (or other parts) for transmitting the power of the motor to the rotary table 142.

The observation-and-imaging unit 200 has a microscope 210 and an imager 220. The microscope 210, which is a so-called inverted optical microscope configured to observe an object from below, includes a stage 211, an illuminator 212 having a light source (e.g., LED) and located above the stage 211, an objective lens (not shown) located below the stage 211, an eyepiece 213 located obliquely upward from the stage 211 on the front side, and an optical system (not shown) configured to receive light passing through the objective lens and bend its optical path obliquely upward so that the light ultimately reaches the eyepiece 213. In the stage 211, at least an area located immediately beneath the dish 610 should be made of a translucent material, or an opening for allowing the light from the illuminator 212 to pass through should be provided. The imager 220 includes: an optical path splitter including a half mirror (or the like); a focusing lens; and an image sensor such as a CCD sensor or CMOS sensor (these devices are not shown). When the observation and imaging of a cell using the observation-and-imaging unit 200 is to be performed, a dish 610 containing the cell is transferred to a position above the stage 211 in the microscope 210 by the container moving unit 100, and light is cast from the illuminator 212 onto this dish 610. After passing through the dish 610 and the objective lens, the light is divided into two directions by the optical path splitter. One of the resulting beams is guided to the eyepiece 213 via the optical system, while the other beam is guided through the focusing lens to form an image on the light-receiving plane of the image sensor. Consequently, an image of the dish 610 observed from below is taken with the imager 220. It should be noted that the microscope 210 does not always need to be an inverted microscope as in the present embodiment; a so-called upright microscope configured to observe an object from above may also be used. In that case, the imager 220 will take an image of the dish 610 observed from above. Furthermore, the illuminator 212 does not always need to be located above the stage 211 to illuminate the dish 610 from above in the previously described manner; the illuminator 212 can also be located below the stage 211 to illuminate the dish 610 from below.

The suction unit 300 includes: a pipet 310 having a tip end to which a pipet tip (which is hereinafter simply called the “tip”) 431 can be attached; an arm 310 supporting the pipet 310; a suction-and-ejection driver 320 provided with a pump (and other components) for suctioning and ejecting a cell through the pipet 310; and a pipet driver 330 (which corresponds to the “sampling tool moving mechanism”) configured to drive the pipet 310 by moving and turning the arm 311. The pipet driver 330, which has a plurality of driving mechanisms including motors and other components (not shown), is configured to produce a pivot motion of the pipet 310 about a vertically extending pivot shaft 331, or to change the posture, i.e. the tilt to the vertical axis (Z axis), of the pipet 310 by swinging the pipet 310 in a plane orthogonal to the horizontal plane, or to produce a linear motion of the pipet 310 in the axial direction of the tip 431 (which corresponds to the “sampling tool” in the present invention) attached to the pipet. The suction unit 300 can bring the pipet 310 to a position close to the plate replacement unit 400 or a position close to the observation-and-imaging unit 200 by producing a pivot motion of the pipet 310 about the pivot shaft 331 by means of the pipet driver 330. The suction unit 300 further includes a tip-removing mechanism (not shown) for removing the tip 431 from the tip end of the pipet 310.

The plate replacement unit 400 includes: a hollow base 410; a driving shaft (not shown) vertically penetrating the upper surface of the base 410; a plate-shaped placement part 420 attached in a horizontal posture to the upper end of the driving shaft; and a placement table driver (not shown) provided in the base 410, including motors and other components (not shown) for rotating the driving shaft or changing its position in the X-axis and Y-axis directions. On the upper surface of the placement part 420, a multi-well plate 422 having a plurality of wells 421 for collecting cells sampled from the dish 610, and a tip rack 430 in which a plurality of tips 431 are set, can be placed. In the plate replacement unit 400, either the multi-well plate 422 or the tip rack 431 placed on the placement part 420 can be selectively brought to a position close to the suction unit 300 by operating the placement table driver to rotate the driving shaft and thereby rotate the placement part 420.

The controller 500 is responsible for controlling the operations of the container moving unit 100, observation-and-imaging unit 200, suction unit 300 and plate replacement unit 400. It consists of a computer having a CPU, memory, and auxiliary storage device consisting of a hard disk drive or flash memory (or the like). Specifically, for example, it may consist of a personal computer or tablet device. The computer acting as the controller 500 is provided with a display unit 570 consisting of a liquid crystal display (or the like) as well as an operation unit 560 consisting of a mouse, keyboard or touch panel (or the like). As shown in FIG. 2, the controller 500 includes, as its functional blocks, a container moving unit controller 510, observation-and-imaging unit controller 520, suction unit controller 530, plate replacement unit controller 540, and display controller 550. The container moving unit controller 510 further includes, as its sub-functional blocks, a sampling target indication receiver 511, determiner 512, horizontal move controller 513, and rotation controller 514. These functional blocks are embodied by executing, on the CPU of the computer, a predetermined program pre-installed on the same computer.

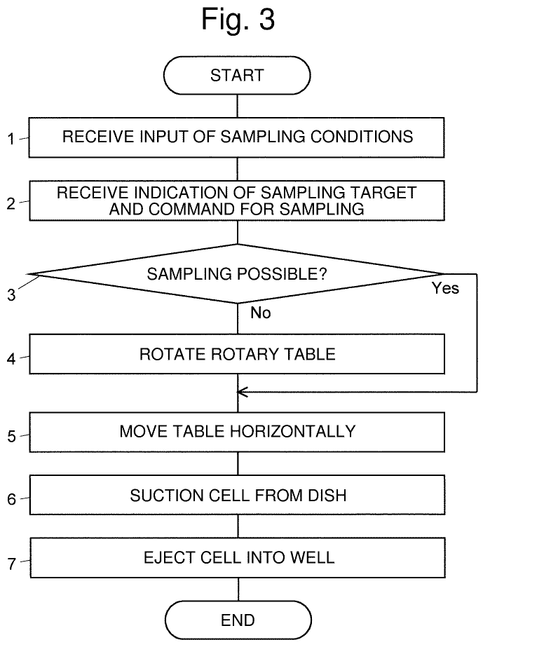

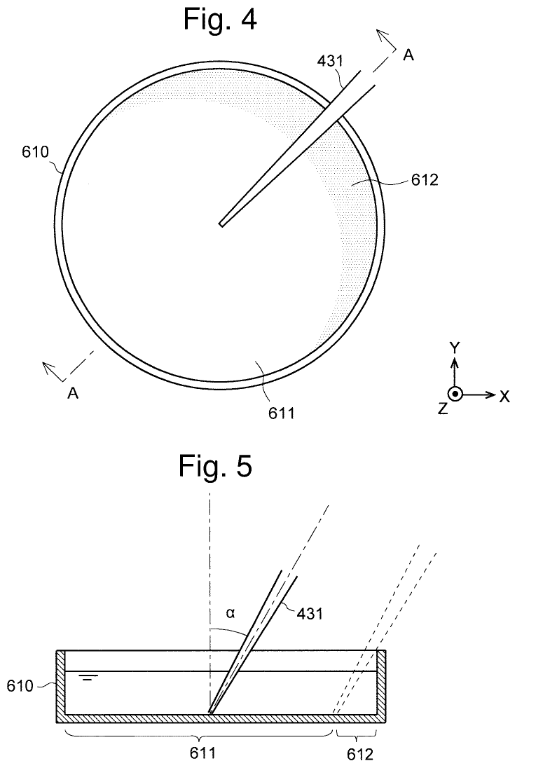

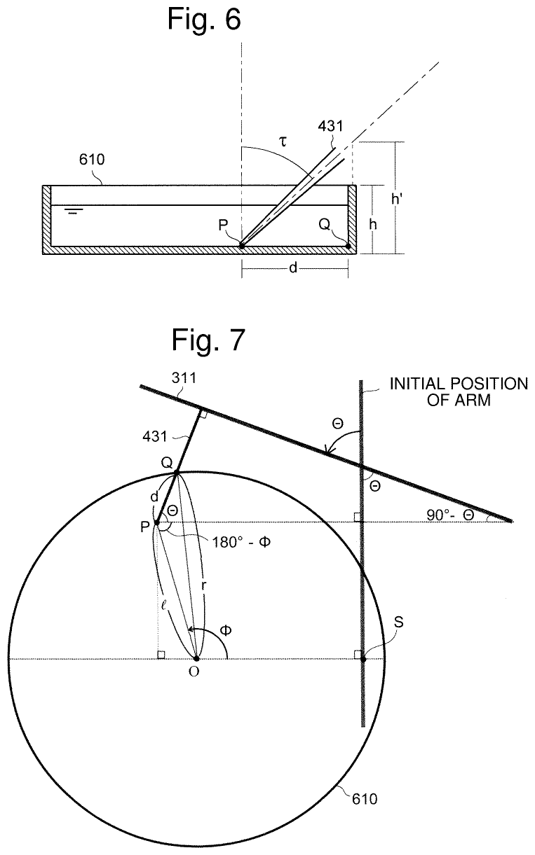

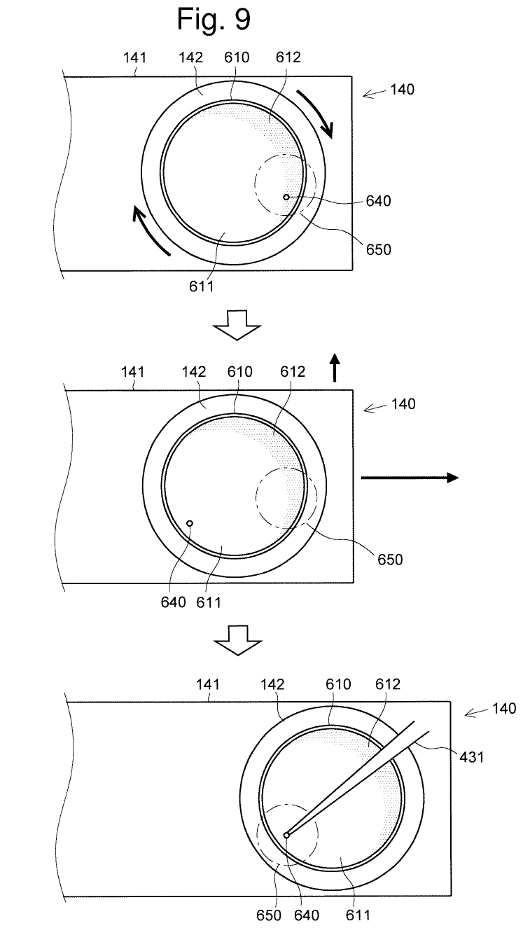

An operation of the cell-picking device according to the present embodiment is hereinafter described with reference to FIGS. 3-9. FIG. 3 is a flowchart showing an operation of the cell-picking device according to the present embodiment. FIG. 4 is a diagram for explaining an arrangeable area and a non-arrangeable area (which will be described later), showing the dish 610 observed from above. FIG. 5 is a sectional view at the arrowed line A-A in FIG. 4. FIGS. 6-8 are diagrams for explaining a method for determining the arrangeable area and the non-arrangeable area. FIG. 9 is a model diagram in which the motions of the table 140 and the rotary table 142 in a cell-sampling process are shown in time series. The area surrounded by the dash-dotted circle 650 in FIG. 9 represents the visual field of the microscope 210.

Initially, the user places the tip rack 430 and the multi-well plate 422 on the placement part 420 in the plate replacement unit 400, and also places the dish 610, which contains cells and a culture medium or similar liquid, at the center of the rotary table 142 in the container moving unit 100. It should be noted that a predetermined type of liquid, such as a culture medium, is previously placed in each well of the multi-well plate 422.

Next, the user operates the operation unit 560 to give a predetermined command to the controller 500. Then, under the control of the display controller 550, a predetermined screen for receiving an input of sampling conditions is shown on the display unit 570 (this screen is hereinafter called the “sampling condition input screen”). On this sampling condition input screen, the user enters the sampling conditions including the diameter of the dish 610, height of the circumferential wall of the dish 610, tilt angle of the pipet 310 in the sampling process, and pivot angle of the pipet 310 in the sampling process (Step 1). The “tilt angle of the pipet 310 in the sampling process” means the tilt (angle α in FIG. 5) of the pipet 310 and the tip 431 to the vertical axis in the process of bringing the tip end of the tip 431 attached to the pipet 310 to a predetermined position on the stage 211 of the observation-and-imaging unit 200 (this position is hereinafter called the “sampling position”). The sampling position is typically, but not limited to, the center of the visual field of the microscope 210. The “pivot angle of the pipet 310 in the sampling process” means the pivot angle around the pivot shaft 331 of the pipet 310 in the process of bringing the tip end of the tip 431 to the sampling position (i.e., the pivot angle of the arm 311 from its initial position). It is possible to have the tilt angle of the pipet 310 in the sampling process and the pivot angle of the pipet 310 in the sampling process previously stored as device-specific values in the controller 500, rather than allowing the user to enter their values in the sampling condition input screen.

The user subsequently performs a predetermined operation with the operation unit 560. Then, the imaging operation using the imager 220 is initiated under the control of the observation-and-imaging controller 520. Consequently, an image taken with the image sensor of the imager 220, i.e., an enlarged image of an inner area of the dish 610, is shown on the display unit 570.

Watching this image, the user performs a predetermined operation with the operation unit 560 to give the controller 500 a command for moving the dish 610 in the right-left direction (X-axis direction) and/or the front-back direction (Y-axis direction). Consequently, under the control of the container moving unit controller 510, the first moving mechanism 120 and/or the second moving mechanism 130 is driven to change the area whose image is taken with the imager 220 within the dish 610.

Watching the image shown on the display unit 570, the user searches for the cell to be sampled. After the cell to be sampled (which is hereinafter called the “sampling-target cell 640”) has been determined, the user performs a predetermined operation with the operation unit 560 (e.g., a tap on the touch panel or a click of the mouse) on the image in which the cell in question shown, to indicate the position of the sampling-target cell 640 (which corresponds to the “sampling target” in the present invention). The user subsequently performs another predetermined operation with the operation unit 560 to give the controller 500 a command for sampling the cell. With this operation, the indication of the sampling-target cell 640 and the sampling command are received by the sampling target indication receiver 511 (Step 2). As described earlier, the rotary table 142 has a recess on its upper surface (i.e., the placement surface), and the position of the dish 610 on the rotary table 142 is fixed by fitting the dish 610 into this recess. Therefore, after the user has indicated the sampling-target cell 640 on the image, the sampling target indication receiver 511 can determine the location of the sampling-target cell 640 within the dish 610 based on the position (coordinates in the X-Y plane) of the rotary table 142, magnification of the microscope 210, position (coordinates) of the sampling-target cell 640 in the image and other related pieces of information at the moment.

Subsequently, the determiner 512 determines whether or not the sampling-target cell 640 can be sampled without rotating the rotary table 142, based on the sampling conditions entered in Step 1 (or previously stored in the controller 500) as well as the position of the sampling-target cell 640 indicated in Step 2 and other related pieces of information (Step 3). Specifically, an area which corresponding to area within the dish 610 as observed from above and which the tip end of the pipet 310 tilted by the aforementioned tilt angle can be brought into by either simply moving the table 140 using the first and second moving mechanisms 120 and 130 or simply moving the pipet 310 using the pipet driver 330, or by combining both moving operations (this area is hereinafter called the “arrangeable area 611”), is initially determined based on the diameter of the dish 610 and the height of the circumferential wall of the dish 610 entered in Step 1, as well as the tilt angle and the pivot angle of the pipet 310 in the sampling process entered in Step 1 or previously stored in the controller 500 as fixed values. It should be noted that the area which corresponds to the inner area of the dish 610 as observed from above, exclusive of the arrangeable area 611, is hereinafter called the “non-arrangeable area 612”. As is evident from FIGS. 4 and 5, the larger the tilt angle of the tip 431 (angle α in FIG. 5) in the sampling process is, the larger the non-arrangeable area 612 becomes. Additionally, the higher the circumferential wall of the dish 610 is, the larger the non-arrangeable area 612 becomes. Furthermore, the location of the non-arrangeable area 612 within the dish 610 changes with the pivot angle.

A method for determining the arrangeable area 611 and the non-arrangeable area 612 is hereinafter described with reference to FIGS. 6-8. FIG. 6 is a sectional view of the dish 610 and the tip 431 at a plane (this plane is hereinafter called “perpendicular plane”) which includes the central axis of the tip 431 and is perpendicular to the X-Y plane which extends at the same level as the inner bottom surface of the dish 610 or at a slightly higher level than the inner bottom surface, with the tip end of the tip 431 located at a predetermined position in the X-Y plane (this position is hereinafter called the “virtual tip-end position P”). In FIG. 6, d represents the length of the perpendicular line drawn from point P to the circumferential wall of the dish 610 in the aforementioned perpendicular plane. Point Q in FIG. 6 represents the intersection point of the perpendicular line and the circumferential wall. In the same figure, h means the height of the circumferential wall of the dish 610, and τ means the tilt angle of the tip 431 to the vertical axis (which corresponds to angle α in FIG. 5). For example, h may be 20 mm, and ti may be 30 degrees. Furthermore, h′ in the same figure means the height from the bottom surface of the dish 610 to the point which is on the central axis of the tip 431 and is directly above the circumferential wall of the dish 610. FIG. 7 is a top view of the dish 610, the tip 431, and the arm 311 supporting the tip 431 under the same condition as in FIG. 6. It should be noted that the tip 431 and the arm 311 in FIG. 7 are simply represented by their respective central axes. In FIG. 7, point O is the center of the dish 610, and r is the radius of the dish 610. For example, r may be 30 mm. Furthermore, l is the length of line segment OP connecting the point O and the virtual tip-end position P, and θ is the angle formed by the central axis of the arm 311 with the tip end of the tip 431 located at the virtual tip-end position P and the central axis of the arm 311 at its initial position. For example, θ may be 70 degrees. It should be noted that the central axis of the arm 311 at its initial position orthogonally intersects with the line extending in the right-left direction through point O, and this point of intersection is hereinafter called “point S”. Additionally, Φ is the angle formed by line segments SO and PO.

As can be understood from FIG. 6, when h′>h, the tip 431 will not collide with the circumferential wall of the dish 610 (i.e., no interference occurs between the tip 431 and the dish 610). Parameter h has a fixed value depending on the dish 610. Therefore, in order to determine whether or not the tip 431 will collide with the circumferential wall of the dish 610 when the tip end of the tip 431 is located at the virtual tip-end position P, only the value of h′ in this situation needs to be calculated.

In order to calculate the value of h′, the value of d needs to be initially determined. From FIG. 7, the angle formed by line segments QP and OP is expressed by θ−Φ+π (where π=180 degrees). According to the law of cosines, r²=l²+d²−2ld cos(θ−Φ+π). Rearranging this equation into a quadratic expression of d gives d²−2l cos(θ−Φ+π)d+l²−r²=0. According to the quadratic formula, d is expressed as follows:

$$d = \frac{2 l cos ( Θ - φ + π ) \pm \sqrt{4 {l}^{2} {cos}^{2} ( Θ - φ + π ) - 4 ( {l}^{2} - {r}^{2} )}}{2} \qquad ( 1 )$$

$$\begin{matrix}d = l cos ( Θ - φ + π ) \pm \sqrt{{r}^{2} - {l}^{2} ( 1 - {cos}^{2} ( Θ - φ + π ) )} & ( 1 )\end{matrix}$$

Since sin²θ+cos²θ=1,

$$d = l cos ( Θ - φ + π ) \pm \sqrt{{r}^{2} - {l}^{2} {sin}^{2} ( Θ - φ + π )} \qquad ( 2 )$$

Since cos(A+π)=−cos A and sin(A+π)=−sin A,

$$d = - l cos ( Θ - φ ) \pm \sqrt{{r}^{2} - {l}^{2} {sin}^{2} ( Θ - φ )} \qquad ( 3 )$$

When l=0, then d=r≥0. Therefore,

$$d = - l cos ( Θ - φ ) + \sqrt{{r}^{2} - {l}^{2} {sin}^{2} ( Θ - φ )} \qquad ( 4 )$$

Since the values of l, θ, Φ and r are known, these values can be substituted into equation (4) to obtain the value of d.

On the other hand, h′ is expressed by the following equation, where it is assumed that τ=30 degrees:

$${h}^{′} = d tan ( \frac{π}{2} - τ ) = d tan ( \frac{π}{2} - 30 ° ) = d tan ( 60 ° ) \qquad ( 5 )$$

The value of h′ can be obtained by substituting the value of d calculated by equation (4) into equation (5).

The value of h′ thus calculated is compared with that of h. If h′>h, it can be concluded that the tip 431 at the virtual tip-end position P will not interfere with the dish 610. Conversely, if h′≤h, it can be concluded that the tip 431 at the virtual tip-end position P will interfere with the dish 610.

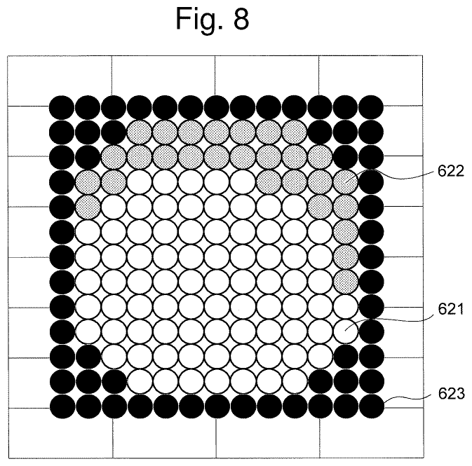

The arrangeable area 611 and the non-arrangeable area 612 described earlier can be determined by varying the virtual tip-end position P in the X-Y plane at the same level as the inner bottom surface of the dish 610 and mapping, on the same X-Y plane, the virtual tip-end positions P at which h′>h and the virtual tip-end positions P at which h′≤h. FIG. 8 shows an example of the result of the mapping. Each white circle 621 in the figure represents a virtual tip-end position P at which h′>h, while each shaded circle 622 represents a virtual tip-end positions P at which h′≤h. In other words, the area within which the white circles 621 are arranged in FIG. 8 corresponds to the arrangeable area 611, while the area within which the shaded circles 622 are arranged corresponds to the non-arrangeable area 612. The area within which the filled circles 623 are arranged corresponds to the area outside the dish 610.

With the arrangeable area 611 (and the non-arrangeable area 612) thus determined, whether or not the sampling-target cell 640 indicated in Step 2 is currently located within the arrangeable area 611 is subsequently determined. In other words, whether or not the cell in question can be sampled without rotating the rotary table 142 is determined (Step 3).

In Step 3, if it has been concluded that the sampling-target cell 640 is located within the arrangeable area 611, the horizontal move controller 513 controls the first and second moving mechanisms 120 and 130 in the container moving unit 100 to move the table 140 in the horizontal plane so that the sampling-target cell 640 is brought to the sampling position (Step 5).

Subsequently, the suction unit controller 530 controls the pipet driver 330 to turn the pipet 310 by the pivot angle entered in Step 1 to the previously specified initial position and to tilt the pipet 310 by the tilt angle entered in Step 1 to the vertical axis. The suction unit controller 530 also controls the pipet driver 330 to bring the tip end of the tip 431 to the sampling position by changing the position of the pipet 310 in the direction toward the tip end of the tip 431 along the axial direction of the tip 431 attached to the pipet 310. With the tip end of the tip 431 attached to the pipet 310 thus located close to the sampling-target cell 640, the suction unit controller 530 subsequently energizes the suction-and-ejection driver 320 to suction the sampling-target cell into the tip 431 (Step 6).

On the other hand, if it has been concluded in Step 3 that the sampling-target cell is not located within the arrangeable area 611 (i.e., the cell is located within the non-arrangeable area 612), the rotation controller 514 rotates the rotary table 142, as shown in the upper section of FIG. 9, to bring the sampling-target cell 640 into the arrangeable area 611 (Step 4; see the upper and middle sections of FIG. 9). In this operation, the rotary table 142 is rotated so that the sampling-target cell 640 is located at a predetermined azimuth about the central axis of the dish 610 as observed from above. For example, this azimuth may be an azimuth having an angular difference of 180 degrees about the central axis from the azimuth of the point at which the tip 431 intersects with the circumferential wall of the dish 610 when the tip end of the tip 431 is located at the center of the dish 610 as observed from above. Selecting such an azimuth enables a more assured sampling of the sampling-target cell. As another possibility, in order to minimize the amount of rotation of the rotary table 142, the rotation angle minimally required for bringing the sampling-target cell 640 into the arrangeable area 611 may be calculated, and the rotary table 142 may be rotated by that angle.

After that, the horizontal move controller 513 controls the first and second moving mechanisms 120 and 130 in the container moving unit 100 to move the table 140 in the horizontal plane so that the sampling-target cell 640 comes to the sampling position (Step 5; see the middle and lower sections of FIG. 9). Then, the suction unit controller 530 controls the pipet driver 330 in the previously described manner to tilt the pipet 310 and change the position of the pipet 310 so that the tip end of the tip 431 attached to the pipet 310 comes to the sampling position (see the lower section of FIG. 9). Subsequently, the suction unit controller 530 energizes the suction-and-ejection driver 320 to suction the sampling-target cell 640 into the tip 431 (Step 6).

After the sampling-target cell 640 has been suctioned into the tip 431 in the previously described manner, the position of the pipet 310, under the control of the suction unit controller 530, is changed in the direction toward the proximal end of the tip 431 along the axial direction of the tip 431 to pull the tip 431 from the dish 610. The pipet 310 is subsequently swung to an upright posture (with the tip end of the tip 431 directed downward) and is also turned around the pivot shaft 331 so that the pipet 310 and the tip 431 come to a position above the placement part 420 of the plate replacement unit 400. Then, under the control of the plate replacement unit controller 540, the placement part 420 is moved and/or rotated in the horizontal plane so that a predetermined well 421 on the multi-well plate 422 placed on the placement part 420 comes to a position directly below the pipet 310. The pipet 310 is subsequently driven toward the tip end of the tip 431 (i.e., downwards) to bring the tip end of the tip 431 close to that well 421, and the suction-and-ejection driver 320 is energized to eject the sampling-target cell 640 from the tip 431 into the well 421 (Step 7).

As described thus far, in the cell-picking device according to the present embodiment, not only can the dish 610 be moved in the horizontal plane by the first and second moving mechanisms 120 and 130, but the dish 610 can also be rotated by the rotation mechanism 143 about the vertical axis passing through the center of the dish. When the sampling-target cell is within the non-arrangeable area, the dish 610 can be rotated to bring the cell into the arrangeable area. Therefore, the cell-picking device according to the present embodiment can avoid the situation in which an area from which cells cannot be sampled occurs within the dish 610. Furthermore, in the cell-picking device according to the present embodiment, after the user has performed the simple operation of indicating the sampling-target cell 640 on the image shown on the display unit 570, the controller 500 determines whether or not that cell is located within the arrangeable area 611. If the cell is not within the arrangeable area 611 (i.e., if the cell is within the non-arrangeable area 612), the controller 500 automatically rotates the rotary table 142 to bring the cell into the arrangeable area 611 before conducting the sampling of the cell. Therefore, the user does not need to be conscious about whether or not the sampling-target cell 640 is within the arrangeable area 611 of the inner area of the dish 610. This reduces the burden of the user associated with the sampling of the cell.

One mode for carrying out the present invention has been described thus far with reference to a specific example. The present invention is not limited to the previously described embodiment; appropriate changes or modifications are allowed within the spirit of the present invention.

For example, in the previous embodiment, the user was allowed to select one sampling-target cell 640 on the image shown on the display unit 570, and the process of sampling (i.e., the suction into the tip 431) and collecting (i.e., the ejection into the well 421 on the multi-well plate 422) that single cell was subsequently performed. Alternatively, the user may be allowed to select a plurality of sampling-target cells on the image, and the previously described process of sampling and collecting one cell may be performed for each of the plurality of sampling-target cells selected. In that case, the tip 431 attached to the pipet 310 should preferably be replaced with a new tip every time the sampling and collection of one sampling-target cell 640 has been completed. After a plurality of sampling-target cells 640 have been selected by the user in the previously described manner, the determiner 512 should preferably determine whether or not each of the sampling-target cells is within the arrangeable area 611 based on the location of each sampling-target cell 640 within the dish 610. Based on the result of this determination, the container moving unit controller 510 should preferably determine the sampling order of the cells so that the total of the amount of rotation of the rotary table 142 and that of the amount of move of the table 140 in the right-left and front-back directions will be minimized. This method can reduce the amount of time for the sampling and collection of a plurality of cells.

In the previous embodiment, whether or not the sampling-targe cell 640 was located within the arrangeable area 611 was determined by the determiner 512, and the rotary table 142 was automatically rotated so as to bring the cell into the arrangeable area 611 when it was concluded that the cell was not located within the arrangeable area 611. Alternatively, whether or not the sampling-targe cell 640 is located within the arrangeable area 611 may be judged by the user, and when it has been concluded that the cell is not located within the arrangeable area 611, the user may manually operate the table or enter instructions into the rotation controller 514 via the operation unit 560 to rotate the rotary table 142 so that the sampling-target cell 640 is brought into the arrangeable area 611.

The previously described embodiment was an example in which the present invention was applied in a cell-picking device. However, the present invention is not limited to devices used for sampling a cell, such as a cultured cell or microbial cell; it is generally applicable in a wide range of picking devices for sampling particulate objects, such as spores, pollens or seeds.

In the previous embodiment, a cell (sampling target) contained in the dish 610 was collected into a predetermined well 421 on the multi-well plate 422. The container used for holding a sampling target before the sampling does not need to be a cylindrical container having an open top and a closed bottom, as with the dish 610. Any type of container may be used as long as it has an opening through which a sampling tool, such as the tip 431, can be inserted. In the case of using a non-cylindrical container, the user should enter other items of information representing the shape of the container in addition to or in place of the diameter and the height of the circumferential wall of the container in the previously described Step 1, and those items of information should be considered when determining the arrangeable area 611 in Step 3. The container for collecting the sampling target obtained with the sampling tool, such as the tip 431, is also not limited to the wells 421 on the multi-well plate 422; any type of container may be used as long as it has an opening through which the sampling tool can be inserted. For example, microtubes may be used as this type of container. In that case, the microtubes should be held in an upright position in a predetermined rack and placed on the placement part 420 in the plate replacement unit 400.

In the previous embodiment, a sampling target in a liquid held in a container (specifically, a cell in a culture medium held in the dish 610) was sampled by suctioning it into the tip 431. However, the picking device according to the present invention is not limited to the configuration in which a tubular tool similar to the tip 431 is used as the sampling tool for sampling the sampling target. For example, the picking device according to the present invention may be configured to scrape a sampling target (e.g., a cell colony) present on a solid (e.g., an agar medium) contained in the dish 610 by means of a rod-shaped sampling tool, such as a platinum loop, to collect the sampling target adhered to the tip end of the sampling tool. It should be noted that the placement table on which the container is to be placed does not need to be horizontal in the case of sampling a sampling-target present on a solid in a container in the previously described manner. This means that the placement surface in the present invention is not limited to a horizontal surface. Similarly, the rotation axis of the placement table in the present invention is not limited to a vertically extending axis; it only needs to extend perpendicularly to the placement surface.

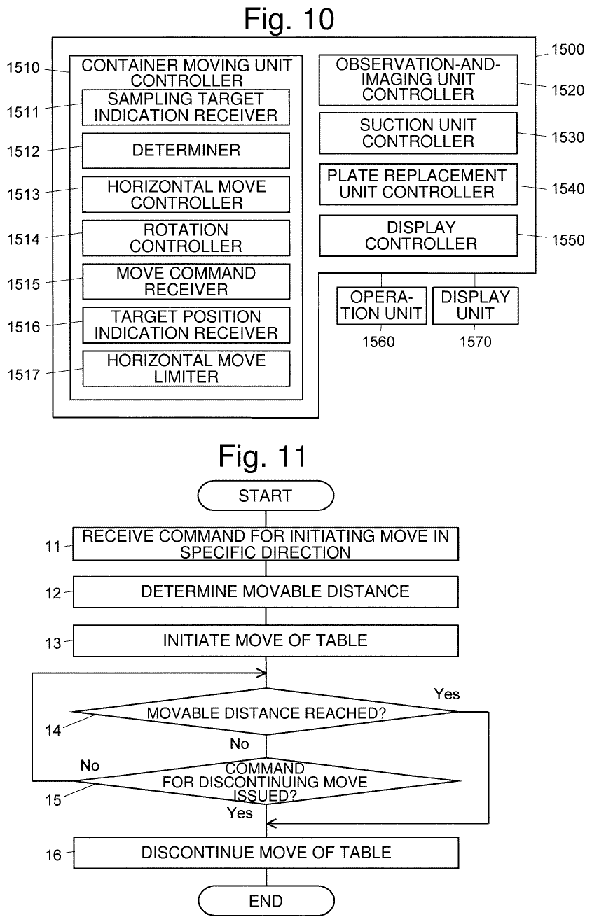

Furthermore, the picking device according to the present invention may be provided with the function of limiting the movable area of the table 140 to a range within which no interference occurs between the sampling tool and the container in the situation where the tip end of the sampling tool (e.g., the tip 431) is inserted in the container (e.g., the dish 610). Such an embodiment is hereinafter described with reference to FIG. 10. FIG. 10 is a block diagram showing the configuration of the main components of the controller 1500 included in the cell-picking device according to the present embodiment. In FIG. 10, the components which have identical or corresponding counterparts in FIG. 2 are denoted by reference signs whose last three digits are common to both figures, and descriptions of those components will be appropriately omitted. It should be noted that the external appearance of the picking device according to the present embodiment as well as its configuration except the controller 1500 are omitted in the drawing since they are substantially identical to those shown in FIG. 1.

The container moving unit controller 1510 in the controller 1500 includes a move command receiver 1515, target position indication receiver 1516 and horizontal move limiter 1517 (which corresponds to the placement table move limiter in the present invention) in addition to the functional blocks similar to those shown in FIG. 2. The move command receiver 1515 is configured to receive commands issued by the user for initiating the move of the table 140 in a specific direction and for discontinuing the move. The target position indication receiver 1516 is configured to receive an indication of the target position of the table 140 by the user. The horizontal move limiter 1517 is configured to limit the movable area of the table 140 to a range within which no interference occurs between the tip 431 and the dish 610 in the situation where the tip end of the tip 431 is inserted in the dish 610.

A procedure for moving the table 140 based on the commands from the user through the move command receiver 1515 in the picking device according to the present embodiment is hereinafter described with reference to the flowchart of FIG. 11. It is hereinafter assumed that the tip end of the tip 431 is inserted in the dish 610 as shown in FIG. 5 or 6.

In this situation, the user performs a predetermined operation with the operation unit 1560 to issue a command for moving the table 140 in a specific direction, and this command is received by the move command receiver 1515 (Step 11). In this step, for example, the move command receiver 1515 may display, via the display controller 1550, a plurality of arrows (or the likes) pointing to different directions on the screen of the display unit 1570 along with the image taken with the imager 220, on which the user selects one of the arrows, whereupon the move command receiver 1515 receives the command for initiating the move in the direction indicated by the selected arrow.

After the command for the moving in the specific direction has been received in the previously described manner, the horizontal move limier 1517 determines the movable distance in that specific direction (Step 12). The “movable distance” means the distance over which the table 140 can be moved from the current position in the specific direction without causing a collision between the tip 431 and the dish 610. For example, the movable distance can be determined by calculating the distance from the current position of the tip end of the tip 431 to the non-arrangeable area 612 in the specific direction.

After the movable distance has been determined, the operation for moving the table 140 in the specific direction is initiated under the control of the horizontal move controller 1513 (Step 13). During the move of the table 140, the horizontal move limiter 1517 determines, at predetermined intervals of time, whether or not the distance over which the table 140 has been moved since the beginning of the move has reached the movable distance (Step 14). If it has been concluded that the movable distance has been reached (i.e., if “Yes” in Step 14), the horizontal move limiter 1517 commands the horizontal move controller 1513 to discontinue the move of the table 140 (Step 16). Conversely, if it has been concluded that the movable distance has not yet been reached (i.e., if “No” in Step 14), the horizontal move limiter 1517 subsequently determines whether or not the command for discontinuing the move from the user has been issued (Step 15). If it has been concluded that the command for discontinuing the move has been issued (i.e., if “Yes” in Step 15), the horizontal move limiter 1517 commands the horizontal move controller 1513 to discontinue the move of the table 140 (Step 16). The command for discontinuing the move is issued, for example, by the user pressing a specific button displayed on the screen of the display unit 1570 in addition to or in place of the aforementioned arrows (or the likes). Another possibility is that the command for initiating the move is entered when the user has touched the arrows (or the likes) with a finger, and the command for discontinuing the move is entered when the user has removed the finger from the arrows (or the likes). As yet another possibility, the container moving unit 100 or the observation-and-imaging unit 200 may be provided with physical buttons for indicating the direction of the move and issuing the command for initiating the move as well as a physical button for issuing the command for discontinuing the move in place of the previously described software buttons.

Conversely, if it has been concluded in Step 15 that the command for discontinuing the move has not yet been received (i.e., if “No” in Step 15), the operation returns to Step 14 to continue the move of the table 140 in the specific direction until the result of Step 14 or Step 15 becomes “Yes”.

As described thus far, in the picking device according to the present embodiment, after the move of the table 140 in the specific direction has been initiated in response to a command for the moving received via the move command receiver 1515, the horizontal move limiter 1517 discontinues the move of the table 140 by the horizontal move controller 1513 either at the point in time when the movable distance has been reached or at the point in time when the command for discontinuing the move has been received by the move command receiver 1515, whichever earlier. Thus, in the situation where the tip end of the tip 431 is inserted in the dish 610, the table 140 can be safely moved within a range in which no interference occurs between the tip 431 and the dish 610.

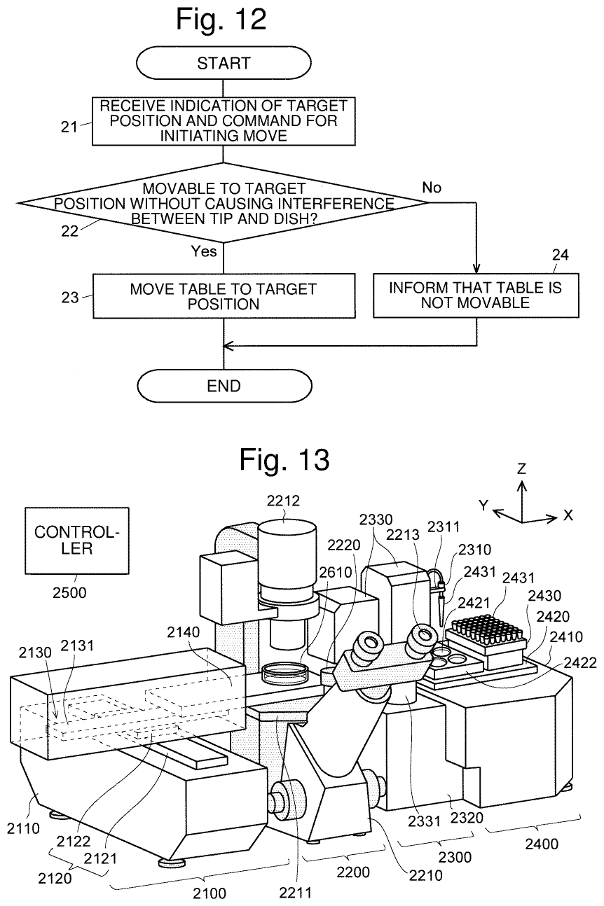

A procedure for moving the table 140 based on a command from the user via the target position indication receiver 1516 in the picking device according to the present embodiment is subsequently described with reference to the flowchart of FIG. 12. Once again, it is hereinafter assumed that the tip end of the tip 431 is inserted in the dish 610 as shown in FIG. 5 or 6.

In the previously described situation, the user initially performs a predetermined operation with the operation unit 1560. Then, the target position indication receiver 1516 displays, via the display controller 1550, a predetermined operation screen on the screen of the display unit 1570, the operation screen allowing the user to indicate the destination (i.e., the target position) of the table 140 and enter the command for initiating the move (Step 21). For example, this operation screen may be configured to allow the user to enter the coordinates of the target position in the X-Y plane, or to allow the user to perform a clicking or tapping operation for indicating a predetermined position on an image of the current visual field of the microscope 210 taken with the imager 220 and displayed on the screen of the display unit 1570. In the latter case, the target position indication receiver 1516 determines, as the target position, the coordinates of the position of the table 140 at which the position indicated by the user comes to the center of the visual field of the microscope 210, based on the coordinates of the current position of the table 140, coordinates of the position indicated by the user in the image, current magnification of the microscope 210, and other related pieces of information.

After the command for the moving in the specific direction has been received in the previously described manner, the horizontal move limiter 1517 determines whether or not the table 140 can be moved to the target position without causing interference between the tip 431 and the dish 610 (this is hereinafter simply described as “movable”; Step 22). For example, this determination in Step 22 can be made based on whether or not the target position is included within the arrangeable area 611.

If the determination result in Step 22 has been “movable” (i.e., if “Yes” in Step 22), the table 140 is moved to the target position under the control of the horizontal move controller 1513 (Step 23). Conversely, If the determination result in Step 22 has been “not movable” (i.e., if “No” in Step 22), the move of the table 140 to the target position is not carried out, and a message informing of the situation is shown on the screen of the display unit 1570 (Step 24).

As described thus far, in the picking device according to the present embodiment, when the move command has been received via the target position indication receiver 1516, the horizontal move limiter 1517 determines whether or not the table 140 can be moved to the target position without causing interference between the tip 431 and the dish 610, and avoids carrying out the move of the table to the target position if the determination result has been “not movable”. Thus, in the situation where the tip end of the tip 431 is inserted in the dish 610, the move of the table 140 can be limited to a range within which no interference occurs between the tip 431 and the dish 610.

The operations of the picking device according to the present embodiment, except for those described using the flowcharts of FIGS. 11 and 12, are substantially identical to the operations in the embodiment shown in FIGS. 1-9. Therefore, descriptions of those operations are omitted.

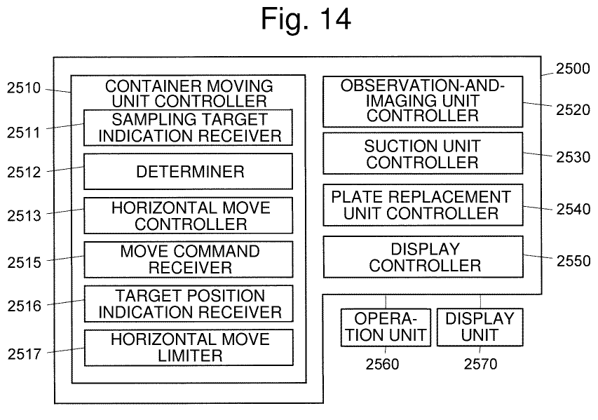

A picking device with no rotary table 142 can also adopt the previously described function of limiting the movable area of the table 140 to a range within which no interference occurs between the sampling tool and the container in the situation where the tip end of the sampling tool (e.g., the tip 431) is inserted in the container (e.g., the dish 610). FIG. 13 shows an external appearance of a picking device according to this type of embodiment, and FIG. 14 shows the configuration of the main components of the controller 2500 in this picking device. In these figures, the components which have identical or corresponding counterparts shown in FIGS. 1, 2 and 10 are denoted by reference signs whose last three digits are common to all of those figures. The configuration of the picking device according to the present embodiment is identical to the configuration shown in FIGS. 1, 2 and 10 except for the absence of the rotary table 142 and the rotation mechanism 143 (see FIG. 1) as well as the rotation controller 514 or 1514 (see FIGS. 2 and 10). Therefore, detailed descriptions of the configuration are omitted. As for the operations of the picking device according to the present embodiment, the operations related to the move of the table 2140 with the tip end of the tip 2431 inserted in the dish 2610 are identical to those shown in the flowcharts of FIGS. 11 and 12, while the other operations are identical to those shown in FIGS. 3-9. Therefore, the descriptions of those operations are omitted.

### [Modes]

It is evident to a person skilled in the art that the previously described illustrative embodiments are specific examples of the following modes of the present invention.

(Clause 1) A picking device according to one mode of the present invention includes:
a placement table having a placement surface on which a container is to be placed;
a placement table rotation mechanism configured to rotate the placement table about a rotation axis which is a virtual axis orthogonal to the placement surface and passing through the center of the placement surface;
a rod-shaped or tube-shaped sampling tool having a tip end configured to be inserted into the container, in a tilted posture to the rotation axis; and
a sampling tool moving mechanism configured to move the sampling tool.

In the picking device according to Clause 1, the placement table can be rotated about the rotation axis passing through the center of the placement surface. Therefore, the situation can be avoided in which an area from which the sampling target cannot be sampled occurs within a container placed on the placement surface.

(Clause 2) The picking device according to Clause 2, which is a picking device according to Clause 1, further includes:
a placement table moving mechanism configured to move the placement table in a plane parallel to the placement surface;
an imager configured to take an image of the container from above or below;
a display section configured to display an image of an inner area of the container taken with the imager;
a sampling target indication receiver configured to receive an indication of a sampling target by a user on the image of an inner area of the container displayed on the display section;
a determiner configured to determine whether or not the sampling target is within an arrangeable area which is an area which the tip end of the sampling tool can be brought into by either moving the placement table by means of the placement table moving mechanism or moving the sampling tool by means of the sampling tool moving mechanism, or by combining both moving operations, without causing interference between the sampling tool and the container; and
a rotation controller configured to drive the placement table rotation mechanism so as to bring the sampling target into the arrangeable area when it is concluded by the determiner that the sampling target is not within the arrangeable area.

In the picking device according to Clause 2, the user indicates the sampling target on an image displayed on the display section, and the determiner determines whether or not that sampling target is within the arrangeable area. If the determination result is that the sampling target indicated by the user is not within the arrangeable area, the rotation controller rotates the placement table to bring the sampling target into the arrangeable area. Therefore, the user does not need to be conscious about whether or not the sampling target is within the arrangeable area inside the container. This reduces the burden of the user associated with the sampling of the sampling target.

(Clause 3) The picking device according to Clause 3, which is a picking device according to Clause 1, further includes:
a placement table moving mechanism configured to move the placement table in a plane parallel to the placement surface; and
a placement table move limiter configured to limit the area within which the placement table can be moved by the placement table moving mechanism, to a range within which no interference occurs between the sampling tool and the container in the situation where the tip end of the sampling tool is inserted in the container by the sampling tool moving mechanism.

(Clause 4) The picking device according to Clause 4, which is a picking device according to Clause 3, further includes a move command receiver configured to receive a command for initiating the move of the placement table in a specific direction and a command for discontinuing the move, where:
the placement table move limiter is configured to determine the distance over which the placement table is movable in the specific direction without causing interference between the sampling tool and the container and to initiate the move of the placement table in the specific direction by means of the placement table moving mechanism when the command for initiating the move is received by the move command receiver, as well as to discontinue the move of the placement table at a later point in time which is either the point in time when the aforementioned movable distance has been reached or the point in time when the command for discontinuing the move is received by the move command receiver, whichever earlier.

(Clause 5) The picking device according to Clause 5, which is a picking device according to Clause 3, further includes a target position indication receiver configured to receive an indication of a target position of the placement table by the user, where:
the placement table move limiter is configured to determine whether or not the move of the placement plate to the target position is possible without causing interference between the sampling tool and the container by moving the placement table by means of the placement table moving mechanism when the indication of the target position is received by the target position indication receiver, as well as to carry out the move of the placement table to the target position by means of the placement table moving mechanism if it is concluded that the move is possible, and to prevent the move if it is concluded that the move is impossible.

(Clause 6) A picking device according to Clause 6 includes:
a placement table having a placement surface on which a container is to be placed;
a placement table moving mechanism configured to move the placement table in a plane parallel to the placement surface;
a rod-shaped or tube-shaped sampling tool having a tip end configured to be inserted into the container, in a tilted posture to an axis perpendicular to the placement surface;
a sampling tool moving mechanism configured to move the sampling tool; and
a placement table move limiter configured to limit the area within which the placement table can be moved by the placement table moving mechanism, to a range within which no interference occurs between the sampling tool and the container in the situation where the tip end of the sampling tool is inserted in the container by the sampling tool moving mechanism.

(Clause 7) The picking device according to Clause 7, which is a picking device according to Clause 6, further includes a move command receiver configured to receive a command for initiating the move of the placement table in a specific direction and a command for discontinuing the move, where:
the placement table move limiter is configured to determine the distance over which the placement table is movable in the specific direction without causing interference between the sampling tool and the container and to initiate the move of the placement table in the specific direction by means of the placement table moving mechanism when the command for initiating the move is received by the move command receiver, as well as to discontinue the move of the placement table at a later point in time which is either the point in time when the aforementioned movable distance has been reached or the point in time when the command for discontinuing the move is received by the move command receiver, whichever earlier.

(Clause 8) The picking device according to Clause 8, which is a picking device according to Clause 6, further includes a target position indication receiver configured to receive an indication of a target position of the placement table by the user, where:
the placement table move limiter is configured to determine whether or not the move of the placement plate to the target position is possible without causing interference between the sampling tool and the container by moving the placement table by means of the placement table moving mechanism when the indication of the target position is received by the target position indication receiver, as well as to carry out the move of the placement table to the target position by means of the placement table moving mechanism if it is concluded that the move is possible, and to prevent the move if it is concluded that the move is impossible.

### REFERENCE SIGNS LIST

100 . . . Container Moving Unit
120 . . . First Moving Mechanism
130 . . . Second Moving Mechanism
140 . . . Table
142 . . . Rotary Table
143 . . . Rotation Mechanism
200 . . . Observation-and-Imaging Unit
300 . . . Suction Unit
310 . . . Pipet
330 . . . Pipet Driver
400 . . . Plate Replacement Unit
500 . . . Controller
510 . . . Container Moving Unit Controller
511 . . . Sampling Target Indication Receiver
512 . . . Determiner
513 . . . Horizontal Move Controller
514 . . . Rotation Controller
570 . . . Display Unit
610 . . . Dish
611 . . . Arrangeable Area
612 . . . Non-Arrangeable Area
420 . . . Multi-Well Plate
431 . . . Pipet Tip
40 . . . Sampling-Target Cell

1. A picking device, comprising:
a placement table having a placement surface on which a container is to be placed;
a placement table rotation mechanism configured to rotate the placement table about a rotation axis which is a virtual axis orthogonal to the placement surface and passing through a center of the placement surface;
a rod-shaped or tube-shaped sampling tool having a tip end configured to be inserted into the container, in a tilted posture to the rotation axis; and
a sampling tool moving mechanism configured to move the sampling tool.

2. The picking device according to claim 1, further comprising:
a placement table moving mechanism configured to move the placement table in a plane parallel to the placement surface;
an imager configured to take an image of the container from above or below;
a display section configured to display an image of an inner area of the container taken with the imager;
a sampling target indication receiver configured to receive an indication of a sampling target by a user on the image of the inner area of the container displayed on the display section;
a determiner configured to determine whether or not the sampling target is within an arrangeable area which is an area which the tip end of the sampling tool can be brought into by either moving the placement table by means of the placement table moving mechanism or moving the sampling tool by means of the sampling tool moving mechanism, or by combining both moving operations, without causing interference between the sampling tool and the container; and
a rotation controller configured to drive the placement table rotation mechanism so as to bring the sampling target into the arrangeable area when it is concluded by the determiner that the sampling target is not within the arrangeable area.

3. The picking device according to claim 1, further comprising:
a placement table moving mechanism configured to move the placement table in a plane parallel to the placement surface; and
a placement table move limiter configured to limit an area within which the placement table can be moved by the placement table moving mechanism, to a range within which no interference occurs between the sampling tool and the container in a situation where the tip end of the sampling tool is inserted in the container by the sampling tool moving mechanism.

4. The picking device according to claim 3, further comprising:
a move command receiver configured to receive a command for initiating a move of the placement table in a specific direction and a command for discontinuing the move,
wherein:
the placement table move limiter is configured to determine a distance over which the placement table is movable in the specific direction without causing interference between the sampling tool and the container and to initiate the move of the placement table in the specific direction by means of the placement table moving mechanism when the command for initiating the move is received by the move command receiver, as well as to discontinue the move of the placement table at a later point in time which is either a point in time when the aforementioned movable distance has been reached or a point in time when the command for discontinuing the move is received by the move command receiver, whichever earlier.

5. The picking device according to claim 3, further comprising:
a target position indication receiver configured to receive an indication of a target position of the placement table by the user,
wherein:
the placement table move limiter is configured to determine whether or not a move of the placement plate to the target position is possible without causing interference between the sampling tool and the container by moving the placement table by means of the placement table moving mechanism when the indication of the target position is received by the target position indication receiver, as well as to carry out the move of the placement table to the target position by means of the placement table moving mechanism if it is concluded that the move is possible, and to prevent the move if it is concluded that the move is impossible.

6. A picking device, comprising:
a placement table having a placement surface on which a container is to be placed;
a placement table moving mechanism configured to move the placement table in a plane parallel to the placement surface;
a rod-shaped or tube-shaped sampling tool having a tip end configured to be inserted into the container, in a tilted posture to an axis perpendicular to the placement surface;
a sampling tool moving mechanism configured to move the sampling tool; and
a placement table move limiter configured to limit an area within which the placement table can be moved by the placement table moving mechanism, to a range within which no interference occurs between the sampling tool and the container in the situation where the tip end of the sampling tool is inserted in the container by the sampling tool moving mechanism.

7. The picking device according to claim 6, further comprising:
a move command receiver configured to receive a command for initiating a move of the placement table in a specific direction and a command for discontinuing the move, wherein:
the placement table move limiter is configured to determine a distance over which the placement table is movable in the specific direction without causing interference between the sampling tool and the container and to initiate the move of the placement table in the specific direction by means of the placement table moving mechanism when the command for initiating the move is received by the move command receiver, as well as to discontinue the move of the placement table at a later point in time which is either a point in time when the aforementioned movable distance has been reached or a point in time when the command for discontinuing the move is received by the move command receiver, whichever earlier.

8. The picking device according to claim 6, further comprising:
a target position indication receiver configured to receive an indication of a target position of the placement table by the user,
wherein:
the placement table move limiter is configured to determine whether or not a move of the placement plate to the target position is possible without causing interference between the sampling tool and the container by moving the placement table by means of the placement table moving mechanism when the indication of the target position is received by the target position indication receiver, as well as to carry out the move of the placement table to the target position by means of the placement table moving mechanism if it is concluded that the move is possible, and to prevent the move if it is concluded that the move is impossible.
